(19) United States
(12) Patent Application Publication
(10) Pub. No.: US 2003/0004571 A1
(43) Pub. Date: Jan. 2, 2003

(54) External breast prosthesis support

(76) Inventor: Danny R. Pace, Halifax (CA)

Correspondence Address: TROJAN LAW OFFICES, 9250 WILSHIRE BLVD, SUITE 325, BEVERLY HILLS, CA, 90212

(21) Appl. No.: 10/148,345

(22) PCT Filed: Nov. 30, 2000
(86) PCT No.: PCT/CA00/01408

(30) Foreign Application Priority Data
Dec. 1, 1999 (CA) 2,291,332

(51) Int. Cl. A61F 2/52

(52) U.S. Cl. 623/7

(57) ABSTRACT

An external breast prosthesis support means comprising a support member attached or attachable at one end to the prosthesis and remotely applicable to the body surface such as to laterally support the weight of the prosthesis. In a preferred embodiment the applicable portion of the support member has a flat undersurface with a high coefficient of static friction against human skin such as to laterally support the weight of the prosthesis and may be overlain on a flat outer surface by a bra shoulder strap such as to transmit load from the shoulder strap downwardly onto the support means, thereby enhancing its load supporting capacity and compensating for sudden increases in load associated with physical activity. The outer surface of the support member has a relatively low coefficient of static friction so as to inhibit the bra shoulder strap from transferring its load laterally to the support means. The invention establishes a natural balance of loads between the body and the bra shoulder strap without requiring attachment of the prosthesis to the anterior body wall, and thereby can be employed much sooner after surgery than conventional prosthesis attachment means. The invention also permits the prosthesis to be worn with a wide range of unmodified bras and to move naturally during vigorous physical activity without shifting.

### FIELD OF INVENTION

The invention pertains to the field of prosthetic devices and, specifically, to means allowing the weight of an external breast prosthesis to be supported, at least in part, by the body surface of a wearer and at a location remote from the prosthesis and, thereby, to provide for a natural balance of loads between the body and a bra sooner than conventional prosthesis attachment means that involve attachment at the site of a mastectomy would comfortably allow.

### BACKGROUND TO THE INVENTION

External breast prostheses are worn by mastectomy patients to restore balance in appearance and weight. Breast prostheses are generally intended to mimic human breasts in form and weight. Breast prostheses are most commonly inserted in bra cups such that the bra supports and secures the prosthesis against the anterior body wall. Unlike natural breasts, which contribute to their own support, breast prostheses, in their simplest common configuration, require that the bra accommodate their entire weight. Prostheses in the correct weight range can place an unnatural load on one side of the bra and are prone to displacement during physical activity. These problems are commonly reduced or overcome by various means. Bras with pockets incorporated into the cups to further support the prosthesis against physical displacement are available (for example, U.S. Pat. No. 5,354,337, Canadian Patent No. CA 1223401), but these do not address the problem of weight load on the bra. Prostheses employing various adhesive means to removably attach them, directly or indirectly, to the anterior body wall are also commonly available (for example, Canadian Patent No. CA 2082992 and U.S. Pat. No. 5,071,433). As the adhesive means must overcome a load that is substantially normal to the body surface, the bonding means must be of high strength and reliability. The direction of load can cause discomfort during physical activity. Also, the nature of load and the application procedures commonly preclude use of such attachment means for some time following surgery.

### SUMMARY

The present invention pertains to an external breast prosthesis support means allowing a breast prosthesis, preferably worn with a bra, to support a portion of its own weight from a position on the body that is remote from the site of a mastectomy. In a preferred embodiment the support means consists of a generally flat band, coplanar with the body surface and extending vertically from the upper border of the prosthesis along the anterior body wall and over the shoulder of the wearer. The support band consists, on its undersurface, of a material which exhibits a high coefficient of static friction against human skin and thereby resists displacement lateral to the skin sufficiently to substantially overcome the weight of the prosthesis. The efficiency of support is further enhanced by placing the bra shoulder strap over the prosthesis support band such that load on the bra shoulder strap is applied normal to the underlying prosthesis support band, securing the support band more firmly against the skin and thereby instantly compensating for rapid increases in load, as may be encountered during physical activity. The outer surface of the prosthesis support band exhibits a low coefficient of static friction against material comprising a bra shoulder strap so as to prevent the bra shoulder strap from applying significant lateral force to the prosthesis support band when load increases on the bra. As the breast prosthesis is not attached to the anterior body wall it can be worn much sooner after surgery than conventional attached prostheses. The invention inhibits shifting of the prosthesis during physical activity and permits the prosthesis to be worn with a wide range of unmodified bras.

### DRAWINGS

The invention will now be described with reference to the following drawings in which:

FIG. 1 shows a perspective view of an external breast prosthesis with attached support means of the present invention.

FIG. 2 shows a vertical section of the upper body of a mastectomy patient, in the shoulder area, with a prosthesis and support means of the present invention installed with a conventional bra.

FIG. 3 shows a sectional view of a support member of the present invention.

FIG. 4 shows a vertical section of the upper body of a mastectomy patient, in the shoulder area, with a prosthesis and support means of the present invention displaying features of alternate embodiments.

### DESCRIPTION OF THE INVENTION

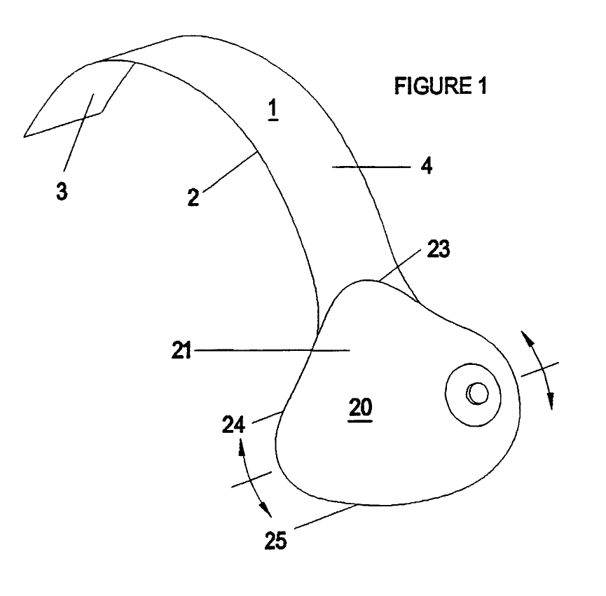

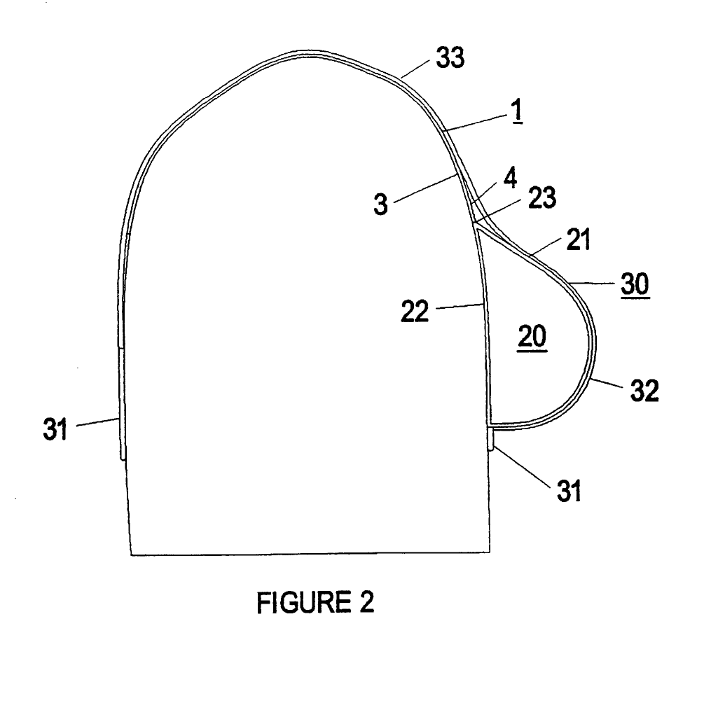

As shown in FIGS. 1 and 2 the invention consists of a support means 1 for an external breast prosthesis 20. The breast prosthesis typically possesses an outer surface 21 that is characteristic in shape of a human breast, and an undersurface 22 that is worn against the body and a border 23 where the outermost surface 21 and undersurface 22 meet to enclose the content substance of the prosthesis. The border 23 typically comprises an upwardly oriented region 24 where a line directed normal to and away from the prosthesis in the plane of the anterior body wall would be directed upwardly, in whole or in part, when the prosthesis is correctly worn, and a downwardly directed region 25 where a line directed normal to and away from the prosthesis in the plane of the undersurface would be directed downwardly, in whole or in part.

In the preferred embodiment the support means 1 consists of a flat, flexible support band 2 that is attached at one end to a portion of the upwardly oriented border region 24 of the prosthesis 20, in the region of the vertical centre line of the prosthesis 2, such that it is generally coplanar with the border 23 of the prosthesis 20, in the area of attachment, and has a major axis that is directed generally upward and away from the prosthesis 20. In the preferred embodiment the support band is of sufficient length to extend to and continue, at least, on to the shoulder of the wearer when the prothesis is installed. The undersurface 3 of the support band 2, at least in the region that is applied to the body surface of the wearer, is comprised of a material, such as silicone rubber, that exhibits a relatively high coefficient of static friction against human skin and the outer surface 4 of the support band 2 is comprised of a material, such as a fabric, that exhibits a relatively low coefficient of static friction against fabric.

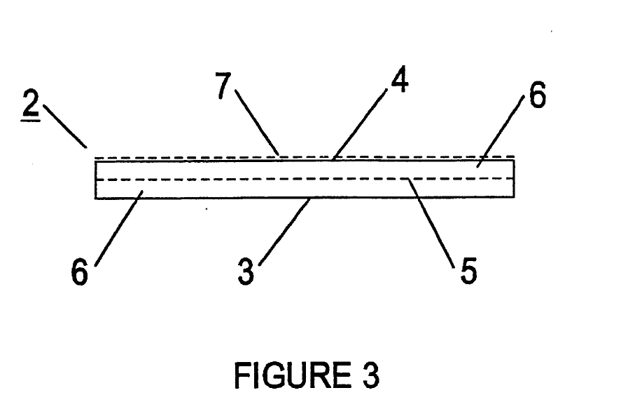

With reference to FIG. 3, and in the preferred embodiment, the support band 2 is comprised of a light, sheer strengthening material 5, such as nylon fabric, sandwiched between two thin layers of clear silicone rubber 6, and on the outermost surface 4, away from the body, overlain, at least in the shoulder region, with a layer of light, sheer and, or skin toned fabric 7, such as to exhibit the required load carrying capacity and frictional characteristics while being inconspicuous under light clothing. In the simplest embodiment, as shown in FIG. 2, and where the support band 2 is of the same material as the prosthesis 20 the support band may be fabricated into the prosthesis 20 so as to provide a smooth and inconspicuous transition from prosthesis 20 to the support band 2. Alternatively, as where the support band and prosthesis are of dissimilar materials, the support band 2 may be bonded to the prosthesis 20 so as to achieve the same transitional effect.

The following alternate embodiments of individual features of the invention may be preferred, for reasons of economy, convenience or comfort, either together or in various combinations with features of the proposed preferred embodiment.

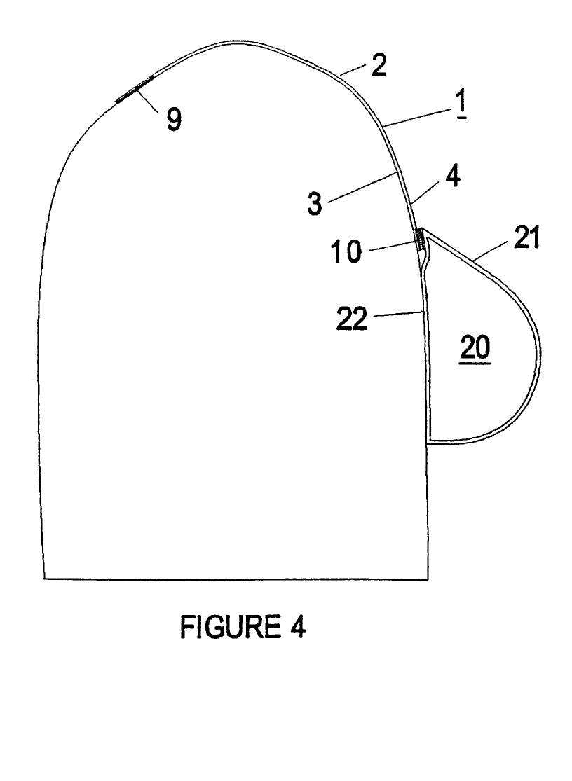

In circumstances where it is not desired or is not practical to wear a bra with a breast prosthesis or where static friction does not provide sufficient support, means may be employed to bond the support band 2, directly or indirectly, to the skin. For example, with reference to FIG. 4, the undersurface 3 of the support band 2 may bear an adhesive or tacky coating 9 that allows the support band 2 to be bonded directly to the body surface at a position remote from the prosthesis such as to support that portion of the weight of the prosthesis that is not supported by other means. For frequent detachment a removable fastener, such as a common hook and loop fastener, bonded at one end to the skin and at the other end to the support band 2, could alternatively be employed.

In an alternate embodiment (FIG. 4) the support band 2 may be removably attached to the breast prosthesis 20, or to an appendage or extension thereof, as by hook and loop fastener 10, thereby permitting the prosthesis 20 to be worn either with or without the support band 2 and the support band 2 to be separately replaced, if necessary.

With regard to the support band 2 it is also understood that other shapes, orientations and attachment sites on the prosthesis than described herein may be employed to achieve a more suitable application of the invention.

### APPLICATION OF THE PREFERRED EMBODIMENT

With reference to FIGS. 1 and 2, the breast prosthesis 20, with attached support means 1, is preferably worn with a conventional bra 30 comprising a horizontal chestband 31, a pair of cups 32 attached along the upper edge of the front portion thereof and a pair of shoulder straps 33 extending upwardly from the cups 32 and over the shoulders of the wearer to the back portion of the chestband 31 so as to transmit downwardly directed load on the cups to the shoulders of the wearer and the back portion of the chestband.

The breast prosthesis 20 is positioned in a bra cup 32 such that the support band 2 lies against the body surface of the wearer and generally in line with the associated bra shoulder strap 33 such as to partially transmit the load generated by the weight of the prosthesis laterally to the body surface in the shoulder region of the wearer by way of frictional resistance between the body surface and the undersurface 3 of the support band 2, and to permit the bra shoulder strap 33 to support the portion of load not supported by the support band 2. The low resistance of the outermost surface material 7 of the support band 2 inhibits the load on the bra shoulder strap 33 from being transmitted laterally to the support band 2. With proper placement of the support band 2 and, or, adjustment of the bra shoulder strap 33, a comfortable balance of loads between the support band 2 and bra shoulder strap 33 is achieved. The bra shoulder strap 33 applies a portion of its load normal to the outer surface of the support band 2, thereby pressing the underlying portion of the support band 2 against the body surface and amplifying resistance of the support band 2 to lateral movement relative to the body surface. In this way the support band 2 is able to compensate instantly for sudden increases in load, as may be experience during physical activity.

While the overall invention has been described in conjunction with the embodiments thereof it is evident that many alternatives, modifications and variations will be apparent to those skilled in the art in light of the foregoing description. Accordingly, it is intended to embrace all such alternatives, modifications and variations as fall within the spirit and broad scope of the appended claims.

1. Support means extendable generally upwardly from an external breast prosthesis and remotely attachable to the body surface of the wearer so as to bear the weight of the prosthesis, in whole or in part.

2. The support means of claim 1 being permanently attached at one end thereof to the said breast prosthesis.

3. The support means of claim 2 having a generally planar undersurface, remotely located from the breast prosthesis, that exhibits a sufficiently high coefficient of static friction against the body surface of the said wearer as to overcome, at least in part, load forces on the said support means generated by the weight of the said prosthesis.

4. The support means of claims 3 having an outermost surface, at least in the region overlain by a bra shoulder strap, that exhibits a low coefficient of static friction against the material of the said bra shoulder strap such as to substantially inhibit said overlying bra shoulder strap from transmitting load forces laterally to the underlying said support means.

5. The support means of claim 1 being removably attached, directly or by intervening attachment means, at one end to said breast prosthesis.

6. The support means of claim 5 having a generally planar undersurface, remotely located from the breast prosthesis, that exhibits a sufficiently high coefficient of static friction against the body surface of the said wearer as to overcome, at least in part, load forces on the said support means generated by the weight of the said prosthesis.

7. The support means of claims 6 having an outermost surface, at least in the region overlain by a bra shoulder strap, that exhibits a low coefficient of static friction against the material of the said bra shoulder strap such as to substantially inhibit said overlying bra shoulder strap from transmitting load forces laterally to the underlying said support means.

8. The support means of claim 2 having an underlying surface remotely located from the said prosthesis and with an adhesive property such as, when applied to the body surface, to overcome load forces on the said support means generated by the weight of the said prosthesis.

9. The support means of claim 5 having an underlying surface remotely located from the said prosthesis and with an adhesive property such as, when applied to the body surface, to overcome load forces on the said support means generated by the weight of the said prosthesis.

10. The support means of claim 2 having, at a position remotely located from the prosthesis, a removable means of attaching the said support means to the body surface such as to overcome load forces on the said support means generated by the weight of said prosthesis and said removable means comprising a fastening means bondable at one end to the body surface and at the other end to the said support means thereby allowing the main body of the said support means to be removably connected.

11. The support means of claim 5 having, at a position remotely located from the prosthesis, a removable means of attaching the said support means to the body surface such as to overcome load forces on the said support means generated by the weight of said prosthesis and said removable means comprising a fastening means bondable at one end to the body surface and at the other end to the said support means thereby allowing the main body of the said support means to be removably connected.
